(19) United States
(12) Patent Application Publication
(10) Pub. No.: US 2006/0045860 A1
(43) Pub. Date: Mar. 2, 2006

(54) Zinc Zeolite Based Deodorants and Deodorizers

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 10/711,136

(22) Filed: Aug. 26, 2004

(51) Int. Cl. A61K 8/27; A23K 1/165

(52) U.S. Cl. 424/67; 424/442

(57) ABSTRACT

This invention relates to Zinc Zeolite based deodorant or deodorizing compositions useful for human or animal body or hair deodorizing solution, deodorizing powder, deodorizing gel, deodorizing spray, deodorizing stick, deodorizing roll-on, deodorizing paste, deodorizing cream, deodorizing lotion, deodorizing aerosol; human or animal deodorizing dentifrice, or oral cavity deodorizing toothpaste, deodorizing mouthwash, deodorizing dental powder, deodorizing mouth spray, deodorizing dental gel, deodorizing lozenges; household deodorizing solution, deodorizing powder, deodorizing gel, deodorizing spray, carpet deodorizer, room deodorizer, and other commonly marketed human, animal, or household deodorizing compositions.

The control of malodor has been of commercial importance since ancient times. A wide variety of solid, liquid and gaseous functional materials including body deodorants, antiperspirants, air fresheners, room deodorants, herbicides, antiviral compositions, fungicides, bactericides, insecticides, depilatory compositions, bleach compositions, hard surface cleaning compositions, skin cleansing compositions, antimicrobial and antifungal preparations, hair setting compositions, hair conditioning compositions, detergent compositions, soap compositions, sunscreen compositions, fabric stain-removal compositions, fabric conditioning compositions, fabric anti-wrinkle compositions, skin lightening compositions, steam iron aroma compositions, candle compositions, plant growth regulating compositions, plant growth stimulating compositions, fertilizer compositions, insect attractant compositions, insect repelling compositions, and drain cleaning compositions have been developed that, although useful for their respective purposes, on use thereof frequently emanate odors which are offensive to the human sense of smell. In addition, a number of defined three-dimensional spaces the use of which is required for various business and service operations and personal matters including indoor gymnasiums, indoor sporting event arenas, locker rooms, hair salons, nail salons, tanning salons, beauty salons, tattoo parlors, pig pens, chicken coops, cow barn enclosures, horse barn enclosures, indoor fresh fish markets, plant processing factory rooms, clothing dry cleaning rooms, garment laundry interiors, rooms containing in-use animal litter containers, abattoirs, cattle cars, zoo animal pens, morgues, autopsy rooms, lavatories, medical patient care rooms, hospital wards and dental patient care rooms have prevailing odors which are offensive to the human sense of smell. This point has been further illustrated in U.S. Patent Application 20040156742 (Milan et al.).

Such odors which are offensive to the human sense of smell are caused by aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, alkali metal sulfites, bisulfites and metabisulfites, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids. Compounds of these classes that have unpleasant odors are referred to herein as malodor compounds. The removal, masking, entrapment, or neutralization of such odors is of significant commercial value. The search for a universal deodorant or deodorizing agent or composition that achieves the sensory perception of the absence of malodor by a combination of all mechanisms including odor entrapment, odor neutralization, and odor masking has not been universally successful in the prior art, as disclosed, for example, in U.S. Pat. No. 6,716,438 (Schroeder et al.); 6,369,026 (Pika et al.); 5,776,475 (Kilpatrick et al.); 5,710,406 (Garris et al.); U.S. Pat. No. 5,665,742 (Mori et al.); and U.S. Pat. No. 5,895,625 (Kawase).

Zeolites are well known as odor absorbing agents for deodorants applications. However, in the presence of water most zeolites lose their deodorizing benefits. This is because zeolites have very small cavities (pores), which trap odor causing molecules. When water is present, then such cavities are filled up with water molecules, leaving no space for the entrance of odor causing molecules. If odor causing molecules have already been trapped in Zeolite cavities, then the addition of water results in the expulsion of odor causing molecules. This is because Na/K zeolites generally have a greater propensity for water molecules over odor causing molecules.

Chemically modified zeolites are also known that have been disclosed to provide certain cosmetic benefits, including deodorization. Examples are noted below.

U.S. Patent Application 20030148876 (Araya) discloses a method of using a zeolite composition that comprises forming a mixture of (a) a crystalline aluminosilicate and (b) a salt of a second metal selected from the group consisting of Group III metals, metallic elements of Group IV, magnesium, titanium, chromium, iron, nickel, copper, zinc, zirconium and silver, said salt of a second metal being present in an amount which is sufficient to replace from about 2.0 to about 40 percent of a first metal moiety, and using said mixture in an aqueous composition at a pH in the range 4 to 10. Such compositions have been claimed to have applications in paper making, paint preparation, dental applications, use of detergents and adsorption and catalytic applications. The aqueous composition used in Araya invention may be a dental composition, for example, in the form of a toothpaste, gel, cream or liquid, of the opaque, translucent or transparent variety. The dental composition contains the zeolite and the salt of a second metal in addition to water and other conventional components of dental compositions. The zeolite in a dental composition will frequently replace conventionally used materials such as silica, chalk and hydrated alumina. Other conventional additives, such as calcium phosphate, calcium pyrophosphate, hydroxyapatites, insoluble metaphosphates etc. may also be present. According to Araya, in view of the known therapeutic properties of zinc compounds, zinc is a useful second metal for use as applied to dental compositions. Araya does not disclose any specific therapeutic benefits for zinc zeolites. Thus, it appears that only the abrasive benefits of zinc zeolites as a replacement for other abrasive agents, such as silica, alumina, and chalk, have been claimed by Araya, and no mention is made for such zinc zeolites providing any deodorancy benefits. U.S. Pat. No. 4,349,533 (Dent et al.) disclose certain pH-adjusted synthetic or natural zeolites, including zinc zeolites, that can be obtained in a particle size range that provides both abrasive cleansing and polishing or lustering of the teeth in the preparation of toothpaste compositions. No deodorizing benefits of zinc zeolites were reported by Dent et al.

U.S. Pat. No. 5,874,067 (Lucas et al.) discloses a method of controlling malodors on human skin comprising the application to the human skin of a composition comprising from about 0.1% to about 5%, by weight of the composition, of solubilized, water-soluble, uncomplexed cyclodextrin; from about 0.1% to about 36%, by weight of the composition, of an oil phase selected from the group consisting of emollients, moisturizers, and skin protectants; an emulsifier; and an aqueous carrier. The compositions may also optionally comprise one or more of the following: hydrophobic antimicrobials; water-soluble antimicrobial preservatives; low molecular weight polyols; zinc salts; water-soluble polymers; soluble carbonate and/or bicarbonate salts; chelating agents; zeolites; activated carbon; and mixtures thereof. The compositions can be applied directly as a spray, poured from a bottle and applied by hand, or applied via a wipe. However, Lucas et al. utilize zeolites and zinc salts in combination with cyclodextrin. No mention is made to zinc zeolites and their application in controlling malodors on human skin. U.S. Patent Application 20020146383 (Johnson) discloses a regimen for hunting that includes using a number of Na/K zeolite-containing personal items in connection with a hunter's apparel and body to reduce the hunter's scent for improved hunting. The personal items include mouthwash, deodorant/antiperspirant, body and boot powder, bar and liquid soap for cleansing the body and hair, laundry detergent and dryer sheets. Each of the personal items includes an effective amount of zeolite, so that use of the personal items reduces the available scent to be detected by animals in the wild. Johnson disclosure only teaches the odor trapping benefits of Na/K zeolites, and no mention is made for the use of zinc zeolite as deodorant ingredients. U.S. Pat. No. 6,730,309 (Horino) discloses a powder comprising a core powder, a hydroxyapatite layered directly on the core, and zinc oxide layered directly on the hydroxyapatite, suitable for use in cosmetics. The powder exhibits good antibacterial effect. The powder is also useful as an effective component for a sebum-adsorbent agent and a body deodorant. No mention is made by Horino as to the preparation or deodorant application of zinc zeolite. However, it appears that the deodorant effect of Horino compositions is most likely due to the antibacterial effect of zinc oxide bound to hydroxyapatite surface. Zinc ions, for example zinc chloride and zinc citrate, are known to impart antibacterial activity, as disclosed in U.S. Patent Applications 20030165439 (De Pierro et al.) and 20030023084 (Burgard). U.S. Patent Application 20030031728 (Martin et al.) discloses antibacterial benefits of silver zinc zeolite. This is not surprising, as silver ions are well known for their strong antibacterial action, for example, U.S. Patent Application 20010009831 (Schink et al.) discloses an antibacterial wound covering comprising a synthetic polymer material comprising zeolites containing silver ions. Nevertheless, Martin et al. do not disclose any deodorant applications of zinc zeolite.

Zinc zeolite itself is well known in prior art, for example, U.S. Patent Application 20010001781 (Drake et al), which relates to a process for the conversion of a cracked gasoline feedstock to ethylene, propylene and BTX (benzene, toluene and xylenes) in the presence of zinc zeolite based catalysts. Drake et al. do not disclose any deodorant applications of zinc zeolite. U.S. Pat. No. 6,357,678 (Hu et al.) and U.S. Pat. No. 6,177,374 (Pradhan et al.) disclose the preparation of zinc zeolite by an ion-exchange process. However, Hu and Pradhan do not disclose any deodorant applications of such zinc zeolites.

Cyclodextrins themselves have been known for a long time for deodorant applications, for example, U.S. Patent Application 20040076591 (Nelson et al.). Cyclodextrins, in combination with other cosmetic ingredients including zinc salts, have been disclosed as deodorants, for example U.S. Pat. No. 6,680,289 and U.S. Patent Application 20040147416 (Woo et al.), U.S. Patent Application 20040127463 (Trinh et al.) and U.S. Patent Application 20030049290 (Jha et al.) relate to a method of removing malodor from fabrics. The composition comprises malodor counteractants such as cyclodextrin, said cyclodextrin being protected from interaction with any other materials that might be present in said composition so as to maintain the cyclodextrin in uncomplexed form and, optionally, zeolites, clay, odor blockers, odor reactants such as aldehydes, essential oil comprising flavanoid, metallic salt, water soluble anionic polymer, etc. to help control odor. U.S. Patent Application 20040157989 (Bruhn et al.) discloses modified cyclodextrins with hollow structures for odor entrapment. Zinc zeolite is not used as a deodorizing or deodorant ingredient in any of these above mentioned disclosures.

U.S. Patent Application 20040002557 (Qian) discloses a dental composition comprising a silver-containing ceramic having antimicrobial and color stabilizing properties and methods for using the composition. The composition comprises a silver-containing glass powder or a silver-containing zeolite powder that may also contain zinc. Zinc zeolite itself and its deodorizing benefits were not claimed by Qian, although antibacterial properties of zinc were mentioned. The antibacterial, anti-irritant, and certain deodorant properties of zinc salts are already well known, for example U.S. Pat. No. 6,774,096 (Paye) discloses anti-irritant benefits of zinc oxide. U.S. Pat. No. 6,528,047 (Arif et al.) discloses a water-soluble home care concentrate that includes a blend of zinc ricinoleate and at least one alkoxylated amine. Arif formulation can be employed in a wide variety of applications including carpet cleaners, bathroom cleaners, pet order removers, cat liter deodorizers, car refresheners, floor cleaners, spray deodorizers, food processing plant cleaners and in other like home care applications. However, Arif et al. do not disclose either zinc zeolite or deodorant applications of zinc zeolite.

In a surprising and unexpected discovery, it has now been found in the present invention that zinc zeolite provides strong deodorant and deodorizing benefits, even in the presence of water. Moreover, a wide variety of compositions can be formulated, which includes deodorant or deodorizing compositions useful for human or animal body or hair deodorizing solution, deodorizing powder, deodorizing gel, deodorizing spray, deodorizing stick, deodorizing roll-on, deodorizing paste, deodorizing cream, deodorizing lotion, deodorizing aerosol; human or animal deodorizing dentifrice, or oral cavity deodorizing toothpaste, deodorizing mouthwash, deodorizing dental powder, deodorizing mouth spray, deodorizing dental gel, deodorizing lozenges; household deodorizing solution, deodorizing powder, deodorizing gel, deodorizing spray, carpet deodorizer, room deodorizer, and other commonly marketed human, animal, or household deodorizing compositions. Zeolites are a group of crystalline aluminosilicates that have a porous structure with a cavity. The preparation and properties of these zeolites are described in detail in U.S. Pat. No. 2,882,243, among other sources. Generally, the preparation involves combining aqueous solutions that are sources of silica, alumina and sodium to produce a gel which crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300° C. provides anhydrous zeolite. Zeolites have several properties that can be highly useful for topical delivery of cosmetic and pharmaceutical compositions. Thus, (a) Zeolites have high adsorptive capacity for water and many organic compounds including toxic metals and enzymes, which makes them useful for many other applications such as water purification, waster water treatment, and chemicals refining/purification; (b) Zeolites are available in certain pore sizes that can be used for self-warming or non-warming cosmetic and pharmaceutical compositions; (c) Zeolites can be made anionic or cationic, which can be used for controlled-release of certain cosmetic and pharmaceutical ingredients via ion-pair mechanisms; (d) Zeolites have a very large surface area that can nearly achieve a nano-particle distribution of organic molecules attached to its vast surface area; (e) Zeolites can also be made in cations other than sodium or potassium; and (f) Zeolites do not absorb into the skin, which is useful for topical delivery of cosmetic and pharmaceutical compositions that are electronically attached to such zeolite surfaces for their controlled or slow delivery over a period of time.

However, many of the prior art applications of zeolites have centered upon their chemical catalysis, heat releasing, or trapping of small molecular weight ingredients. Zeolites also have outer surface area, in addition to such inner pore surface areas. The functional properties of zeolites utilizing both inner and outer surface areas have not been disclosed in the prior art as well.

Zeolites can be made with both specific pore structures and bound cations that have found applications in various self-warming cosmetic compositions. U.S. Pat. No. 3,250,680 (Menkart et al.) discloses applications of Zeolites for the preparation of self-heating toothpaste and other such compositions. This utilizes only the heat releasing property of zeolites. U.S. Pat. No. 4,626,550 (Hertzenberg) discloses certain personal care products such as lotions and creams that are prepared using Zeolite A that contains sodium and potassium.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various problems.

U.S. Pat. No. 6,274,128 (Bergman et al.) discloses an essentially anhydrous hair conditioning composition comprising zeolites of specific pore size larger than the critical diameter of a water molecule and both the carrier molecules and the hair conditioner molecules that have molecular diameters larger than the largest average pore size of the micro porous materials. As is clearly evident, such constraints are not convenient or commercially achievable at a reasonable cost.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect.

U.S. Application 20010016201 (Janchitraponvej) discloses a yet another self-heating application of an anhydrous rinse-out hair care composition utilizing zeolites.

Zeolites can also be made in cations other than sodium or potassium. U.S. Patent Application 20040014591 (Muller et al.) discloses titanium and other transition metal zeolites. U.S. Patent Application 20010021368 (Masini et al.) discloses certain metal zeolites, including zinc zeolite. U.S. Pat. No. 6,357,678 (Hu et al.) discloses preparation of zinc zeolites by a very difficult multi-step process. U.S. Pat. No. 6,605,267 (Lee et al.) discloses process for making metal zeolites with quaternary ammonium compounds, useful as chemical catalysts. U.S. Pat. No. 6,084,142 (Yao et al.) discloses the preparation of a zinc zeolite, and its application in petroleum cracking process. U.S. Pat. No. 6,177,374 (Pradhan et al.) discloses the preparation of silicon, zinc and aluminum zeolites, and their application in petroleum cracking process. Yao and Pradhan do not disclose any deodorant or deodorancy applications of such zeolite derivatives. U.S. Pat. No. 6,479,427 (Anthony at al.) and U.S. Pat. No. 5,502,240 (Pugach) disclose titanium zeolites and their application in petroleum cracking process. U.S. Pat. No. 5,772,917 (Kynast et al.) discloses a cesium zeolite that is luminescent. U.S. Pat. No. 6,106,797 (Muller et al.) discloses titanium or vanadium zeolites useful for accelerating oxidation reactions. U.S. Pat. No. 6,008,389 (Grosch et al.) discloses titanium and vanadium zeolites useful as catalysts for the preparation of epoxides, in particular propylene oxide, from olefins, hydrogen and oxygen. U.S. Patent Application 20030035763 (Vergani et al.) discloses the use of iron and manganese zeolites in the purification of organometallic compounds utilizing such zeolite's adsorptive properties. U.S. Patent Application 20030024856 (Surana et al.) discloses a yet another application of zeolite's adsorptive properties in removing odors. These zeolites do not remove odors in the presence of water, as mentioned herein. U.S. Patent Application 20020127402 (Green et al.) discloses the antimicrobial applications of silver ions attached to zeolites by ion-exchange methods.

It is worthy of note that although zeolites with many different cations, such as titanium, zinc, manganese, iron, quaternary ammonium, and copper have been disclosed, any applications of such metal zeolites for deodorant and deodorizing benefits, with or without the presence of water, have not been disclosed in the prior art.

This lack of prior art knowledge is of special note, since zeolites with enhanced ion-exchange capacity are already well known, for example U.S. Patent Application 20010053741 (Mikko et al.) and U.S. Pat. No. 5,935,891 (Prior). U.S. Pat. No. 6,503,740 (Alther et al.) discloses zeolites treated with an organic modification compound such as quaternary amines, pyridinium compounds, and phosphonium amines that are useful for water treatment applications. U.S. Pat. No. 6,365,130 (Barry et al.) discloses zeolites exchanged with antimicrobial metals, such as silver, for a chewing gum application, or a laundry application (U.S. Pat. No. 6,454,813; Chan). Modified zeolites have been used for topical cancer therapy (U.S. Pat. No. 6,288,045; Kaufman). Additionally, U.S. Pat. No. 4,620,929 (Hofmann) and U.S. Pat. No. 3,935,067 (Thayer) teach the use of expanded clay or plastic materials in combination with bentonite, which advantageously exhibits moisture retention characteristics. However, any applications of such metal zeolites for deodorant and deodorizing benefits, with or without the presence of water has not been disclosed in the prior art.

The present invention discloses surprising and unexpected discoveries that zinc zeolite provides strong deodorant and deodorizing benefits even in the presence of water. Moreover, a wide variety of compositions can be formulated, which includes deodorant or deodorizing compositions useful for human or animal body or hair deodorizing solution, deodorizing powder, deodorizing gel, deodorizing spray, deodorizing stick, deodorizing roll-on, deodorizing paste, deodorizing cream, deodorizing lotion, deodorizing aerosol; human or animal deodorizing dentifrice, or oral cavity deodorizing toothpaste, deodorizing mouthwash, deodorizing dental powder, deodorizing mouth spray, deodorizing dental gel, deodorizing lozenges; household deodorizing solution, deodorizing powder, deodorizing gel, deodorizing spray, carpet deodorizer, room deodorizer, and other commonly marketed human, animal, or household deodorizing compositions.

Although the precise mechanism of this deodorancy benefit of zinc zeolite is still unknown, it is the hypothesis of the present inventor that zinc ions in the zeolite cavity actually help the retention of odor causing molecules when such molecules enter zeolite cavity. Moreover, the presence or the addition of water does not cause such odor molecules to unbind from zeolite cavity and escape, as is the case with sodium and potassium zeolites. It is a further belief of the present inventor that since most odor causing molecules have atoms that contain excess electrons, such as sulfur, phosphorus, or nitrogen, the zinc ion in zinc zeolite being an electron deficient bidentate or divalent ion, can bind with zeolite on one side and excess electrons on sulfur, phosphorus, or nitrogen atoms of the odor causing molecule on the other side, thus holding such odor molecules in a strong ion-pair or donor-acceptor bond within zinc zeolite's cavity and also its outer surface. Water contains oxygen, which also carries excess electrons in its orbital structure, but those excess electrons on oxygen atom of water molecule do not bind as strongly with zinc atom as excess electrons on nitrogen, phosphorus, or sulfur atoms of odor causing molecules. This is quite surprising and unexpected since oxygen atom in water molecule is more electronegative than sulfur, phosphorus, or nitrogen atoms of odor causing molecules. The electronegativity order, for example, is O (3.44)>N (3.04)>S (2.58)>P (2.1)>Zn (1.65). Since most metals such as zinc are essentially electropositive, it is expected that atoms with greater electronegativity will bind more strongly with such electropositive atoms. For this unexpected reason, even the hydrated forms of zinc zeolite show excellent deodorant and deodorizing benefits. The anhydrous forms of zinc zeolite appear to be more effective in deodorant and deodorizing benefits, but that may be due to more zinc zeolite present on a percent weight basis in the anhydrous form than the hydrated form. Additionally, zinc zeolite still maintains excellent antibacterial and antifungal benefits. This is also quite unexpected and surprising since it is known that chelating action usually neutralizes the antibacterial activity of such metal atoms, and zinc is most likely present in a chelated form within the zeolite cavity.

The present invention also incorporates simple in-situ preparation of zinc zeolite and its application in deodorant and deodorizing compositions. Zinc zeolite can be prepared by a very simple process by the ion-pair exchange of a zinc derivative, such as zinc chloride, zinc sulfate, zinc nitrate, zinc acetate, zinc gluconate, zinc EDTA, etc., with a commonly available sodium/potassium zeolite, as illustrated in Equation 1 to 3.
Zinc Chloride+Sodium/Potassium Zeolite→Zinc Zeolite+Sodium (potassium) Chloride (Equation 1).
Zinc Acetate+Sodium/Potassium Zeolite→Zinc Zeolite+Sodium (potassium) Acetate (Equation 2).
Zinc Gluconate+Sodium/Potassium Zeolite→Zinc Zeolite+Sodium (potassium) Gluconate (Equation 3).

It should be noted that zeolites contain sodium and potassium cations that can be exchanged with other cations. For certain metal atoms it is commonly known that the exchange efficiency is in the following order: Ba>Pb>Cd>Zn>Cu>K>Na>Li. The exchange amount is determined by the exchange capacity of such zeolites, which is usually expressed as milli-equivalents (meq) of a cationic composition to per gram weight of zeolite. A zeolite with 1.0 meq per gram exchange capacity, for example, can exchange 0.068 grams of zinc chloride per gram of such zeolite. This is calculated as follows. The molecular weight of zinc chloride is 136.3. Thus, 136.3 grams of zinc chloride equals 1000 milli-equivalents (or, 1 mole equivalent), or 0.136 grams of zinc chloride equals one milli-equivalent. Since each zinc chloride molecule has two chlorine atoms that can undergo exchange, only half the equivalent amount of zinc chloride will thus be needed to exchange monovalent cations (such as sodium or potassium) in that zeolite. Thus, only 0.06815 grams of zinc chloride will be needed to exchange with one gram of zeolite for a complete exchange (i.e. 136.3/1000/2=0.0681 5). In practice, total exchange is not required. Typically, only 10 to 50% of all available monovalent cations need to be exchanged. In another example, 0.0917 grams of zinc acetate (molecular weight 183.4) will be needed to completely exchange one gram of zeolite that has one meq per gram of exchange capacity with two acetate anions to be exchanged (i.e. 183.4/1000/2=0.092).

Moreover, the exchange reactions of the present invention can be carried out either in hydrous or in anhydrous systems. This offers a great advantage for the preparation of anhydrous zeolites containing divalent and polyvalent cations. The preparation of divalent or polyvalent metal zeolites by ion-exchange is usually carried out in an aqueous medium followed by their dehydration at elevated temperatures, during which many divalent metal zeolite cage structures collapse. The methodology of the present invention circumvents this problem and permits the preparation of anhydrous zeolites with divalent or polyvalent cations without requiring a high temperature dehydration step, since anhydrous forms of zeolites can now be exchanged with divalent or polyvalent cations in an anhydrous medium according to the teachings of the present invention.

In actual preparative process, a solution of zinc derivative in water or another solvent or solvent mixture is stirred with zeolite. Zinc zeolite is thus formed by the in-situ process, as shown in Equation 1, 2, and 3.

Zinc zeolite can be formulated in various additional cosmetic and pharmaceutical consumer products utilizing a variety of delivery systems and carrier bases. Such consumer product forms include the group consisting of shampoos, aftershaves, sunscreens, band lotions, skin creams, liquid soaps, bar soaps, bath oil bars, shaving creams, dishwashing liquids, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products, spritzes, styling waxes and balms.

In another preferred aspect, the delivery system or a carrier base are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and similar delivery or packaging form.

In another preferred aspect, the delivery system can be human body or hair deodorizing solution, deodorizing powder, deodorizing gel, deodorizing spray, deodorizing stick, deodorizing roll-on, deodorizing paste, deodorizing cream, deodorizing lotion, deodorizing aerosol, and other commonly marketed human body and hair deodorizing compositions, household deodorizing solution, deodorizing powder, deodorizing gel, deodorizing spray, carpet deodorizer, room deodorizer, and other commonly marketed household deodorizing compositions, animals and pets deodorizing solution, deodorizing powder, deodorizing gel, deodorizing spray, animals and pets carpet deodorizer, animals and pets room deodorizer, and other commonly marketed animal and pet deodorizing compositions.

In another preferred aspect, the cosmetically acceptable composition is selected from the group consisting of shampoos, aftershaves, sunscreens, hand lotions, skin creams, liquid soaps, bar soaps, bath oil bars, shaving creams, dishwashing liquids, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products, spritzes, styling waxes and balms.

In another preferred aspect, the delivery system can be traditional water and oil emulsions, suspensions, colloids, microemulsions, clear solutions, suspensions of nanoparticles, emulsions of nanoparticles, or anhydrous compositions.

Additional cosmetically or pharmaceutically beneficial ingredients can also be included in the formulated compositions of the present invention, which can be selected from, but not limited to skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another preferred aspect, the cosmetically acceptable composition further comprises one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and di-tail phospholipids.

Representative saccharides include nonionic or cationic saccharides such as agarose, amylopectins, amyloses, arabinans, arabinogalactans, arabinoxylens, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomainans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994) which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable composition of this invention may include surface-active agents. Surface active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used in this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metal and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable composition of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate.

Amphoteric surface-active agents which may be used in the cosmetically acceptable composition of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL.™. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable composition of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyidimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyidimonium chloride, Hydroxyethyl tallowdimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryidimonium chloride, cetalkonium chloride, dicetyidimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable compositions may include long chain fatty amines from about C.sub.10 to C.sub.22 and their derivatives. Specific examples include dipalmitylamine, lauramidopropyldimethylamine, and stearamidopropyl dimethylamine. The cosmetically acceptable compositions of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof. Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable composition of the present invention include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable composition of this invention are typically used in an amount from about 0.1 to 50 percent by weight, preferably from about 0.5 to about 40 percent by weight, more preferably from about 1 to about 20 percent by weight.

The cosmetically acceptable composition of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable composition of this invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable composition of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The compositions of this invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane endblocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable compositions of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane. 200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable compositions of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in compositions of the current invention. Preferred examples include dimethicone copolyols and 5225C Formulation Aids, available from Dow Corning, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Corning 8220, Dow Corning 939, Dow Corning 949, Dow Corning 2-8194, all available from Dow Corning, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable compositions of this invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the tradename Permethyl 101A, available from Presperse, South Plainfield, N.J., USA. Another example of a preferred volatile hydrocarbon is C.sub.12 to C.sub.14 isoparaffin, under the tradename Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable compositions of this invention may include cationic and ampholytic conditioning polymers. Examples of such include, but are not limited to those listed by the International Cosmetic Ingredient Dictionary published by the Cosmetic, Toiletry, and Fragrance Association (CTFA), 1101 17 Street, N.W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetically acceptable composition of this invention may include one or more rheological modifiers. The rheological modifiers which can be used in this invention include, but are not limited to high molecular weight crosslinked homopolymers of acrylic acid, and Acrylates/C10-30 Alkyl Acrylate Crosspolymer, such as the Carbopol. and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropylttrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in the cosmetically acceptable composition of this invention at a concentration of from 0.01 to 12 weight percent, preferably from 0.05 to 10 weight percent and most preferably from 0.1 to 6 weight percent.

The cosmetically acceptable composition of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 5 weight percent, preferably 0.1 to 3 weight percent and most preferably from 0.2 to 2 weight percent of the cosmetically acceptable composition.

The cosmetically acceptable composition of this invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis (hydroxypropyl) aminobenzoate, 2-ethylhexy 1-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, and titanium oxide, and mixtures thereof. The amount of sunscreen used in the cosmetically acceptable composition of this invention will vary depending on the specific UV absorption wavelength(s) of the specific sunscreen active(s) used and can be from 0.1 to 10 percent by weight, from 2 to 8 percent by weight.

The cosmetically acceptable composition of this invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis (hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable composition of this invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The surface of pigments, such titanium dioxide, zinc oxide, talc, calcium carbonate or kaolin, can be treated with the unsaturated quaternary ammonium compounds described herein and then used in the cosmetically acceptable composition of this invention. The treated pigments are then more effective as sunscreen actives and for use in color cosmetics such as make up and mascara.

The cosmetically acceptable composition of this invention can be presented in various forms. Examples of such forms include, but are not limited a solution, liquid, cream, emulsion, dispersion, gel, thickening lotion.

The cosmetically acceptable composition of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerine, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. These solvents can be present in proportions of up to as much as 70 percent by weight, for example from 0.1 to 70 percent by weight, relative to the weight of the total composition.

The cosmetically acceptable composition of this invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable composition of this invention also can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

Compositions for treating skin include leave-on or rinse-off skin care products such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, skin care compositions may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The skin care compositions of this invention may be prepared as oil-in-water, water-in-oil emulsions, triple emulsions, or dispersions.

Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

Compositions for treating hair include bath preparations such as bubble baths, soaps, and oils, shampoos, conditioners, hair bleaches, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products. The dispersion polymers may also be utilized in styling type leave-in products such as gels, mousses, spritzes, styling creams, styling waxes, pomades, balms, and the like, either alone or in combination with other polymers or structuring agents in order to provide control and hair manageability with a clean, natural, non-sticky feel.

Hair care compositions of this invention give slippery feel and that can be easily rinsed from the hair due to the presence of the dispersion polymer, volatile silicones, other polymers, surfactants or other compounds that may alter the deposition of materials upon the hair.

In the case of cleansing formulations such as a shampoo for washing the hair, or a liquid hand soap, or shower gel for washing the skin, the compositions contain anionic, cationic, nonionic, zwitterionic or amphoteric surface-active agents typically in an amount from about 3 to about 50 percent by weight, preferably from about 3 to about 20 percent, and their pH is general in the range from about 3 to about 10.

Preferred shampoos of this invention contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The shampoo for washing hair also can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

Other preferred embodiments consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the compositions are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acryinitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients or compositions can be selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

UV inhibitor or UV absorber for color stabilization can be selected from including, but not limited to Kaempferia galanga extract, aminobenzoic acid, Cinoxate, Ethylhexyl methoxycinnamate, Avobenzone, Homosalate, Lawsone, Menthyl anthranilate, Octocrylene, Ethylhexyl salicylate, oxybenzone, Padimate-O, Benzophenone-3, Benzophenone-4, Sulisobenzone, Titanium dioxide, Zinc oxide, Trolamine salicylate, Glyceryl aminobenzoate, and combinations thereof.

The amount of zinc zeolite can be from 0.01% to 100% of composition. This is because zinc zeolite can be used as is in the form of a formulation, for example, as a 100% Zn Zeolite “Super Strength” body deodorant or deodorizing powder (Example 8). However, zinc zeolite can also be added in as small as 0.01% amounts in lipstick type compositions for deodorizing lips (Example 15). This is additionally illustrated in the Examples section of this invention.

### EXAMPLES

The examples cited herein are for illustrative purposes only and do not represent any limitations of the present invention. All percent (%) amounts are by weight basis.

### Example 1

### Preparation of Zinc Zeolite from Zinc Chloride

Ingredients. (1) Zeolite, Type 4A 20.0 (2) Zinc chloride 1.36 (3) Water 78.64. Procedure. Mix (2) and (3) to a clear solution. Add (1) and mix. The mixture contains Zinc zeolite (100% zeolite exchanged), made by the in-situ ion-pair exchange.

### Example 2

### Preparation of Zinc Zeolite from Zinc Acetate

Ingredients. (1) Zeolite, Type 4A 40.0 (2) Zinc Acetate 0.18 (3) Propanol 59.82. Procedure. Mix (2) and (3) to a clear solution. Add (1) and mix. The mixture contains Zinc zeolite (5% zeolite exchanged), made by the in-situ ion-pair exchange.

### Example 3

### Deodorant Powder Composition for Human or Animal Use

Ingredients (1) Zeolite 78.7 (2) Chlorphenesin 0.3 (3) Tetrahydrocurcuminoids 0.2 (4) Boswellin 0.2 (5) Magnolol 0.1 (6) Paeonol 0.5 (7) Sodium Gluconate 10.0 (8) Zinc Zeolite 10.0. Procedure. Mix all ingredients in a ball mill.

### Example 4

### Deodorant Aerosol Composition for Human or Animal Use

Ingredients (1) SD Alcohol 42.0 (2) Aminomethylpropanol 0.38 (3) Acrylates/Octylacrylmide Copolymer 2.0 (4) Zinc Zeolite 10.0 (5) Isobutene (propellant) 45.62. Mix (1) and (2). Slowly add (3) with mixing. Add (4) and mix. Fill cans with the resulting composition and (5).

### Example 5

### Deodorant Roll-On or Spray Composition for Human or Animal Use

Ingredients (1) Water 43.65 (2) Methyl Parabens 0.15 (3) Diazolidinyl Urea 0.2 (4) EDTA 0.05 (5) Acrylates/C10-30 Alkyl Acrylate Crosspolymer 0.3 (6) Triethanolamine 0.3 (7) Ethylhexyl Hydroxystearate 4.0 (8) Propyl Parabens 0.1 (9) Capric/Caprylic Triglyceride 3.25 (10) Witch Hazel Distillate 28.0 (11) Zinc Zeolite 20.0. Procedure. Mix (1) to (6) to a clear solution. Add (7) to (9) and mix at high speed at 40 to 50 C. Cool to 30-35 C and add all other ingredients and mix. Cool to room temperature. Fill in roll-on or spray delivery system packaging.

### Example 6

### Mouth Deodorizing and Triple Whitening Natural Toothpaste Composition for Human or Animal Use

Ingredients (1) Sorbitol 25.0 (2) Sodium Saccharin 0.27 (3) Trisodium Phosphate 1.0 (4) Potassium Cocoate 10.0 (5) Glycerin 10.0 (6) Cellulose Gum 0.3 (7) Water 24.28 (8) Titanium Dioxide 0.95 (9) Silica (fine abrasive) 12.0 (10) Silica (coarse abrasive) 4.0 (11) Peppermint Oil 0.5 (12) Spearmint 0.25 (13) Menthol 0.25 (14) Urea Peroxide 0.5 (15) Calcium Peroxide 0.5 (16) Benzalkonium Chloride 0.2 (17) Zinc Zeolite (10.0). Procedure. Mix (7) and (8) first, then add all other ingredients and mix in a high speed homogenizer.

### Example 7

### Carpet Deodorizing Composition

Ingredients (1) Zinc Zeolite 99.5 (2) Fragrance 0.5. Procedure. Mix both ingredients.

### Example 8

### Refrigerator Deodorizing Composition

Ingredients (1) Zinc Zeolite 100.0. Procedure. Fill zinc zeolite in packaging with small holes.

### Example 9

### Refrigerator Deodorizing Composition with Baking Soda

Ingredients (1) Zinc Zeolite 50.0 (2) Sodium Bicarbonate 50.0. Procedure. Mix both ingredients. Fill in packaging with small holes.

### Example 10

### Body Deodorant Sachet or Stick Composition

Ingredients (1) Zinc Zeolite 20.0 (2) Alkyl Benzoate 49.9 (3) Ethylenediamine/Hydrogenated Dimer Dilinoleate Copolymer Bis-Di-C14-C18 Alkyl Amide 10.0 (4) Exotic Butter Blend (Mango butter, Coco butter, Shea butter) 0.1 (5) Fragrance 20.0. Procedure. Mix (2) to (4) and heat at 70 to 80 C to a clear solution. Cool to 30 to 40 C. Add all other ingredients and mix. Fill in sachet, tube, tub, or stick packaging and cool to room temperature.

### Example 11

### Deodorant Talcum Body Powder

Ingredients (1) Corn Starch 66.0 (2) Talc 14.0 (3) PEG-62.0 (4) Zinc Zeolite 15.0 (5) Tetrahydrocurcumin 0.5 (6) Vitamin K-10.5 (7) Dimethicone 2.0. Procedure. Mix (1) and (2). Premix (3) to (6) and add to main batch and mix. A powder composition is obtained.

### Example 12

### Body Deodorant Stick Composition

Ingredients (1) Sodium Stearate 8.0 (2) Propylene Glycol 7.5 (3) PEG-4 7.5 (4) Cyclomethicone 40.0 (5) Isostearyl Alcohol 19.5 (6) PPG-10 Cetyl Ether 10.0 (7) Zinc Zeolite 7.5. Procedure. Mix (1) to (3) and heat at 70 to 80 C. Cool to 60 to 65 C and add all other ingredients. Mix and pour in stick deodorant packaging. Cool in cooling tunnel.

### Example 13

### Deodorizing Stick Composition

Ingredients (1) PEG-12 Lanolin 4.0 (2) Oleyl Alcohol 2.0 (3) Propylene Glycol 71.0 (4) Sodium Stearate 8.0 (5) Zinc Zeolite 15.0. Procedure. Mix (1) to (4) and heat at 70 to 80 C. Cool to 60 to 65 C and add (5). Mix and fill deodorant stick packaging and cool in cooling tunnel.

### Example 14

### Deodorizing and Warming Facial Mask with Pumice Scrubber

Ingredients (1) Glycerin 46.5 (2) Dimethicone 2.0 (3) Red, Green, Blue Algae Extract 0.8 (4) Phenoxyethanol 0.5 (5) Ethylhexylgicein 0.5 (6) Fragrance 0.2 (7) Zeolite 30.0 (8) Zinc Zeolite 14.0 (9) Sodium Silicoaluminate (Veegum) 2.0 (10) Pumice 3.5. Procedure. Mix all ingredients in a high speed mixing equipment to a paste. Fill in tubes.

### Example 15

### Deodorant Lip Balm Composition

Ingredients (1) Mineral Oil 21.69 (2) Aloe Vera 0.5 (3) Ethylhexyl Palmitate 18.0 (4) Lanolin 2.0 (5) Petrolatum 24.65 (6) Ozokerite Wax 24.65 (7) Beeswax 12.0 (8) Cetyl Alcohol 2.0 (9) Paraffin Wax 10.0 (10) Vitamin E 0.2 (11) Propyl Paraben 0.2 (12) Brown Colorant 0.65 (13) Menthol 2.0 (14) Palmitoyl Pentapeptide 0.1 (15) Zinc Zeolite 0.01. Procedure. Mix (1) to 11) at 70 to 80 C. Cool to 60 to 65 and add all other ingredients. Mix and fill lip balm tubes and cool.

### Example 16

### Deodorant Body Lotion Composition

Ingredients (1) Purified Water 82.7994 (2) Glyceryl Stearate 1.0 (3) Phenoxyethanol 0.9 (4) Glycerin 3.8 (5) Mango oil 0.0001 (6) Pyridoxine 0.0001 (7) Aloe vera 0.0001 (8) Ascorbyl Palmitate 0.0001 (9) Methyl Paraben 0.2 (10) Propyl Paraben 0.1 (11) PGMS-SE 2.0 (12) Stearic Acid 3.0 (13) Cetyl Alcohol 1.2 (14) Carbomer 0.0001 (15) Zinc Zeolite 5.0. Procedure. Mix (1) to (14) and heat at 75 to 85 C. Cool to 45 to 55 C and add all other ingredients. Mix and cool to room temperature.

What is claimed is:

1. A deodorant or deodorizing composition comprising; (a) zinc zeolite, and (b) a delivery system or a cosmetically acceptable carrier base composition.

2. A composition according to claim 1, wherein zinc zeolite can be in hydrated or anhydrous forms.

3. A composition according to claim 1, wherein delivery system or carrier bases can be zinc zeolite itself.

4. The compositions according to claim 1, wherein such compositions contain Zinc Zeolite from 0.01% to 100%.

5. A composition according to claim 1, wherein zinc zeolite can be from an in-situ generated process or in a pre-manufactured form.

6. A composition according to claim 1, wherein delivery system or carrier base can include additional ingredients or compositions selected from skin cleansers, cationic surfactants, anionic surfactants, non-ionic surfactants, amphoteric surfactants, and zwitterionic surfactants, skin and hair conditioning agents, vitamins, hormones, minerals, plant extracts, anti-inflammatory agents, whitening agents, deodorants, odor masking agents, antiperspirants, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

7. A composition according to claim 1, wherein delivery system or a carrier base are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and similar delivery or packaging forms.

8. The compositions according to claim 1, wherein the delivery system can be traditional water and oil emulsions, suspensions, colloids, microemulsions, clear solutions, suspensions of nanoparticles, emulsions of nanoparticles, or anhydrous compositions.

9. The compositions according to claim 1, wherein the delivery system can be human body or hair deodorizing solution, deodorizing powder, deodorizing gel, deodorizing spray, deodorizing stick, deodorizing roll-on, deodorizing paste, deodorizing cream, deodorizing lotion, deodorizing aerosol, and other commonly marketed human body and hair deodorizing compositions.

10. The compositions according to claim 1, wherein the delivery system can be household deodorizing solution, deodorizing powder, deodorizing gel, deodorizing spray, carpet deodorizer, room deodorizer, and other commonly marketed household deodorizing compositions.

11. The compositions according to claim 1, wherein the delivery system can be animals and pets deodorizing solution, deodorizing powder, deodorizing gel, deodorizing spray, animals and pets carpet deodorizer, animals and pets room deodorizer, and other commonly marketed animal and pet deodorizing compositions.

12. The compositions according to claim 1, wherein the delivery system can be human or animal deodorizing dentifrice, or oral cavity deodorizing toothpaste, deodorizing mouthwash, deodorizing dental powder, deodorizing mouth spray, deodorizing dental gel, deodorizing lozenges, and other commonly marketed human or animal deodorizing dentifrice, or oral cavity deodorizing compositions.
